(19) United States
(12) Patent Application Publication
(10) Pub. No.: US 2025/0205008 A1
(43) Pub. Date: Jun. 26, 2025

(54) TAG FOR TRACKING AND NAVIGATION OF A MEDICAL DEVICE

(71) Applicant: Aesculap AG, Tuttlingen (DE)

(72) Inventors: Arne Jansen-Troy, Stockach (DE); Allan Maas, Konstanz (DE); Dennis Goerz, Tuttlingen (DE); Stephan Dorau, Herzogenaurach (DE); Andreas Elisch, Dunningen (DE); Stefan Thomas, Tuttlingen (DE); Matthias Schweizer, Tuttlingen (DE); Matthias Henke, Villingen-Schwenningen (DE)

(21) Appl. No.: 18/849,632

(22) PCT Filed: Mar. 24, 2023
(86) PCT No.: PCT/EP2023/057685

§ 371 Date: Sep. 23, 2024

(30) Foreign Application Priority Data
Mar. 31, 2022 (DE) 10 2022 107 688.1

(51) Int. Cl. A61B 90/00; A61B 90/98

(52) U.S. Cl. CPC A61B 90/39; A61B 90/98

(57) ABSTRACT

A tag for determining the location and/or tracking the location and/or determining the orientation of a medical device includes at least one radio-based tracking unit designed to actively and/or passively communicate wirelessly with a first external device, preferably by means of NFC, Bluetooth Low Energy, DECT or WLAN. The tag has at least one optical marker element that conveys information, optically detectable by at least a second external device, regarding its position and orientation in a virtual coordinate system. A medical device can include the tag and a system for location and/or orientation determination.

### CROSS-REFERENCE TO RELATED APPLICATIONS

This application is the United States national stage entry of International Application No. PCT/EP2023/057685, filed on Mar. 24, 2023, and claims priority to German Application No. 10 2022 107 688.1, filed on Mar. 31, 2022. The contents of International Application No. PCT/EP2023/057685 and German Application No. 10 2022 107 688.1 are incorporated by reference herein in their entireties.

### FIELD

The present disclosure relates to a tag for determining the location and/or tracking the location and/or determining the orientation of a medical product, a medical product configured with the tag, and a system for determining the location and/or tracking the location and/or determining the orientation of a medical product.

### BACKGROUND

In medical care, ever higher demands are being placed on efficiency while at the same time meeting high quality and hygiene requirements. This applies in particular to processes of handling medical products. These processes include, for example, cleaning and sterilization processes as well as storage and transport processes. In particular, the prevention of errors in cleaning and sterilization processes as well as storage and transport processes is essential to ensure patient safety.

These processes can be automated in order to prevent errors in cleaning and sterilization processes as well as storage and transport processes and to increase efficiency at the same time. However, in order to automate the processes reliably and safely, it is necessary to be able to clearly localize the medical product in question.

In the state of the art, RFID tags are used to localize medical products. For example, RFID tags are used in WO/2016 059 382 to check whether an instrument set is complete and to track it.

However, localization using RFID tags is relatively imprecise and does not provide any information about the exact location or spatial orientation of the medical product, meaning that automating handling processes (picking up instruments, moving instruments, placing instruments down, etc.) based on the localization of an instrument using RFID tags entails risks such as collisions or incorrect handling.

### SUMMARY

It is therefore an object of the present disclosure to increase process reliability in automated handling of medical products, in particular process reliability in automated cleaning and sterilization processes of medical products, and thus to improve efficiency and patient safety.

Specifically, the object is solved by a tag for determining the location and/or tracking the location and/or determining the orientation of a medical product with at least one radio-based tracking unit, which is provided and configured to communicate wirelessly with at least one first external device actively and/or passively, preferably via NFC, Low Energy Bluetooth/Bluetooth Low Energy (BLE), DECT or WLAN. Furthermore, the tag has at least one optical marker element which is provided and configured to indicate information that is optically detectable by at least one second external device with respect to its position and orientation in a virtual coordinate system.

In other words, the tag is configured in the form of a recognition element with the radio-based tracking unit, which is configured actively, for example as a beacon/radio beacon, using Bluetooth Low Energy, and/or passively, for example as an RFID tag. The tag is provided and configured to be attached to the medical product. In the active embodiment, the radio-based tracking unit emits a wireless tracking signal that is detectable by the at least one first external device. In the passive embodiment, a wireless tracking signal is emitted by the first external device, which is detectable by the passive radio-based tracking unit. In addition, the tag is configured with the at least one optical marker element. The optical marker element is an optically detectable marking configured on a surface of the tag. The optical marker element is based on a unique virtual coordinate system. The optical marker element can be optically detected/recognized by the at least one second external device. Ideally, the optical marker element is optically configured with high contrast. Furthermore, the optical marker element is configured asymmetrically and is not configured as a data matrix code in order to avoid malfunctions.

By configuring the tag with both the radio-based tracking unit and the optical marker element, determining the location of the tag can be significantly improved. In particular, the radio-based tracking unit enables rough localization, which allows the location to be determined down to several centimeters and ensures, for example, completeness checks of instrument sets. The supplementary optical marker element may be used to determine the location in the millimeter range and thus make the rough location determined by the radio-based tracking unit more precise and validate it by comparing the determined positions. In addition, the optical marker element enables the tag's orientation to be determined spatially.

Furthermore, the combination of the radio-based tracking unit and the optical marker element may considerably accelerate the exact determination of the location of the tag, in particular in comparison to a variant in which only an optical marker element is configured, since the radio-based tracking unit determines an approximate position of the tag and the exact determination of the location can be carried out in a more targeted manner by the optical marker element.

The configuration with both the radio-based tracking unit and the optical marker element also significantly increases the tag's failure safety. In other words, it is still possible to determine the location even if the optical marker element on the surface of the tag is damaged, for example by mechanical impact or corrosion, or if the radio-based tracking unit is damaged or destroyed, for example by a technical defect.

In addition, by configuring the radio-based tracking unit and the optical marker element in a single component, the latter can be flexibly attached to different objects, so that existing medical products can be easily retrofitted with the tag of the present disclosure.

Accordingly, the core of the disclosure is to provide the tag with both a radio-based tracking unit and an optical marker element for determining the location and/or orientation.

In a first aspect, the tag may be configured with at least one machine-readable data element and/or at least one alphanumeric data element.

In other words, the tag may additionally contain a machine-readable data element, for example in the form of a barcode or QR code, and/or a human-readable alphanumeric data element. The machine-readable data element and the alphanumeric data element may, for example, contain information about properties and/or content and/or intended use of the medical product to which the tag may be attached. In particular, the information may be article and serial numbers.

In a further aspect, the tag may include at least one optical signal output device and/or an acoustic signal output device.

In other words, the tag may include a screen and/or an ePaper display and/or indicator lights/indicator LED and/or a speaker and/or a piezo buzzer. Via the optical and/or acoustic signal output device, the tag can output information about the tag, information about the medical product to which the tag is attached, and/or about the tag-medical product relationship.

In a further aspect, the tag may be configured with a through hole in a direction normal to a planar extension of the tag.

In other words, the tag may be configured with a through hole in the form of a window, which enables elements located behind the tag to be shown.

In a further aspect, the tag may be configured with at least one energy generating element, preferably in the form of a photovoltaic element or a piezo element.

In other words, the tag may include a self-sufficient energy supply with the energy generating element and optionally an energy storage/energy buffer, which supplies electrical components with energy. The energy generating element may be a photovoltaic element that converts light energy into electrical energy using solar cells. Alternatively, the energy generating element may be a piezo element, which generates an electrical voltage from an acting mechanical force.

By configuring the tag with the energy generating element, the tag may be operated over long periods of time with little maintenance and without the need for an energy storage unit to be recharged by a user. In particular, it can be ensured that when the tag is configured with an active radio-based tracking unit, this is supplied with electrical energy.

In a further aspect, the tag may be configured with at least one manual status sliding element.

In other words, the tag may be configured with the manual status sliding element in the form of a mechanical sliding element, which can be used to manually make a certain status recognizable. This can be done, for example, by revealing a color and/or text. This allows the user to change the status of the tag manually. For example, a user can use this method to mark a medical product as contaminated or to indicate the filling status of a container, in particular a sterile goods container.

In a further aspect, the tag may be configured with at least one switch, for example a push or slide switch, which enables electronic functions of the tag to be switched on and off.

In a further aspect, the tag may have a plate-shaped, substantially rectangular, in particular a square geometry.

In a further aspect, the tag may include fastening elements, preferably latching hooks, extending perpendicularly away from the plate-shaped geometry.

In a further aspect, the tag may have an asymmetrical geometry.

In a further aspect, the optical marker element may be configured as a relief, which can be detected by a time-of-flight sensor, for example.

In a further aspect, the tag may emit a radio signal which, for example, emits an identifier of the tag and can be received by an external display device, for example on the outside of a cabinet, so that it can be indicated when the tag is inside the cabinet.

The object of the present disclosure is furthermore solved by a medical product according to claim 7. Advantageous further embodiments are part of the associated dependent claims.

The medical product is preferably a sterile goods container which is provided and configured to store, transport and aseptically provide sterile instruments and the like at a location where the sterile instruments are used. Furthermore, the sterile goods container may be provided and configured to safely store and transport used and thus contaminated instruments and the like and to protect a user from contamination.

The medical product includes a tag according to one of the preceding embodiments and is configured with a tag receptacle that receives the tag in a predetermined position and orientation with respect to the medical product or parts thereof.

In other words, the medical product is configured with the tag, wherein the tag is fixed in the tag receptacle. The tag receptacle is provided and configured to receive the tag in exactly one predetermined position and orientation, relative to an outer geometry of the medical product or relative to individual parts of the medical product, such as a handling portion, a gripping portion, an opening of the medical product or the like, in a force-fitting and/or form-fitting and/or firmly bonded manner. In yet other words, the tag and the tag receptacle are matched to each other according to the poka-yoke principle.

In this way, it can be ensured that the tag is only connected to the medical product in a predetermined position and orientation. This means that the marker element arranged on the tag and the radio-based tracking unit are also arranged and oriented in a predetermined position and orientation in relation to the outer geometry of the medical product or in relation to individual parts of the medical product, such as the handling portion or the gripping portion or the opening or the like. This ensures, for example, that an external handling device can reliably determine a position of the handling portion via the radio-based tracking unit and the marker element in order to be able to handle the medical product in a positionally reliable manner.

Preferably, the tag is configured on a side facing the tag receptacle with latching geometries, for example in the form of latching hooks, which engage in corresponding recesses in the tag receptacle. The poka-yoke principle may be configured via an arrangement of the latching geometries and a corresponding arrangement of the recesses in the tag receptacle.

In an alternative embodiment, the receiving geometry may be configured in an aperture which is connected to the medical product in a firmly-bonded, form-fitting and/or force-fitting manner. The aperture can preferably be an essentially plate-shaped element.

In a further aspect, a disconnecting switch may be configured between the tag and the tag receptacle of the medical product, which is provided and configured to detect a disconnection of the tag from the medical product.

In other words, the tag and/or the tag receptacle may be configured with the disconnecting switch. The disconnecting switch detects when the tag moves further than a predetermined distance from the tag receptacle. The predetermined distance may preferably be a few millimeters. The disconnecting switch may, for example, be configured as a pressure switch, a proximity sensor, or a magnetic switch or the like.

In a further aspect, the medical product or the tag may output an optical and/or acoustic alarm signal when the disconnecting switch detects the disconnection, i.e. when the predetermined distance between the tag and the tag receptacle is exceeded. The output may, for example, take place via the optical and/or acoustic signal output device of the tag.

The disconnecting switch, which detects the disconnection between tag and tag receptacle, may, in particular in combination with the alarm output, prevent a collision of the handling device with the medical product due to an incorrect arrangement and orientation relationship between the at least one marker element arranged on the tag and the radio-based tracking unit and the handling portion of the medical product. Furthermore, the disconnection of the tag from the medical product can be easily and quickly detected by an operator/user, so that it is easier to assign the tag to the medical product after an unintentional disconnection.

In a further aspect, the medical product may include, preferably in the tag, a disconnect signal transmitter unit that transmits a wireless disconnect signal when the disconnecting switch detects the disconnection of the tag from the medical product.

In other words, the disconnect signal transmitter unit may transmit the wireless disconnect signal when the predetermined distance between the tag and the tag receptacle is exceeded.

Preferably, the wireless disconnect signal is received by an external control unit, which then notifies the operator and/or stops the handling device, for example. In this way, it can be ensured that a possible collision between the handling device and the medical product and thus possible damage to the handling device and/or the medical product and its contents is prevented.

The object of the present disclosure is further solved by a system according to claim 10. Advantageous further embodiments are part of the associated dependent claims.

The system for determining the location and/or tracking the location and/or determining the orientation of a medical product consists of, inter alia:
the tag according to one of the preceding embodiments,
the at least one first external device, which wirelessly communicates actively and/or passively with the tag of the medical product and determines a position of the tag based on a range and/or a signal strength of at least one wireless signal and/or based on lateration of the wireless signal, in particular based on trilateration or multilateration, and
the at least one second external device, which optically detects a position and an orientation of the tag via the at least one optical marker.

In other words, the system includes the tag configured on a medical product, wherein the tag includes the radio-based tracking unit and the optical marker element. Furthermore, the system includes the first external device, which is provided and configured to actively or passively detect the radio-based tracking unit of the tag and to determine the position of the tag based, for example, on a range and/or a signal strength of the signal between the first external device and the radio-based tracking unit. Preferably, methods established in positioning technology are used for this purpose, such as Bluetooth Low Energy, RFID, DECT, W-LAN or something similar. Trilateration or multilateration may also be used to improve position determination. The system also includes the second external device, which detects the at least one optical marker on the tag. The detection takes place optically, for example via at least one camera or via at least one laser or the like. In other words, the second external device may be a camera or a laser. In the event that the optical marker is a relief, the second external device may be a time-of-flight sensor (TOF camera) or ultrasonic sensor.

In one aspect, the system may include at least one handling device provided and configured to grip and handle the medical product at a handling portion.

In other words, the system may include at least one handling device, for example in the form of a robot arm, a conveyor belt or another actuator, which is provided and configured to move the medical product (in a predetermined/defined manner). All spatial directions are conceivable as directions of movement. Preferably, the medical product is force-fittingly and/or form-fittingly connected to the handling device during the movement process.

In a further aspect, the system may include a processing unit that is provided and configured to determine a position of the handling portion from the detected position of the tag based on geometric dependencies stored in the processing unit.

In other words, the system may include a processing unit that determines the position of the handling portion in particular from the position of the tag determined by the first external device and the second external device. For this purpose, the predetermined geometric dependencies, for example the exact positional orientation of the tag receptacle or the tag with the at least one optical marker and the radio-based tracking unit in relation to the handling portion in a virtual coordinate system, may be stored in the processing unit. These can be entered once by a user or made available by the manufacturer of the medical product.

In a further aspect, the system may include a comparison device that compares a first position signal determined by the first external device with a second position signal determined by the second external device.

In other words, the system may include the comparison device provided and configured to validate the position of the tag determined by the first external device using the position of the tag determined by the second external device.

In a preferred embodiment, if the comparison device detects a deviation of the determined positions exceeding a limit value, an optical and/or acoustic signal may be output and/or a stop error may be output, which is suitable for stopping the handling device.

Such a comparison device may prevent false detections of the first external device and/or the second external device from causing a collision between the handling device and the medical product.

### BRIEF DESCRIPTION OF THE DRAWINGS

FIG. 1 is a representation of a sterile goods container with a front plate and a tag;

FIG. 2 is an enlarged representation of the tag on the front plate of the sterile goods container;

FIG. 3 is a schematic representation of the tag in a plan view;

FIG. 4 is a sectional view along line B-B;

FIG. 5 is another schematic representation of the tag in a side view; and

FIG. 6 is another schematic representation of a rear side of the tag.

### DETAILED DESCRIPTION

Configuration examples of the present disclosure are described below on the basis of the associated Figures.

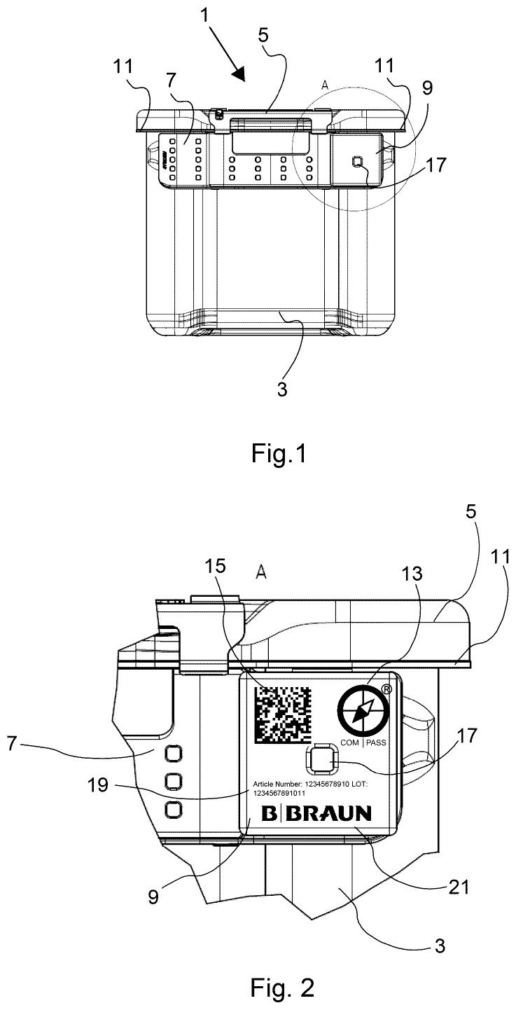

FIG. 1 shows a medical product in the form of a sterile goods container 1. The sterile goods container 1 comprises a container body 3 and a lid 5, which is provided and configured to close an opening of the container body 3. A front plate 7 is firmly bonded and/or form-fittingly attached to a portion of the container body 3 facing the lid 5. The front plate 7 contains a tag receptacle (not shown). The tag receptacle is provided and configured to receive a tag 9 form-fittingly and/or force-fittingly. In other words, the tag 9 is fixed in position and orientation on the front plate 7. A handling portion 11 is configured on the lid 5. Alternatively, the handling portion 11 may also be configured directly on the container body 3. The handling portion 11 is a geometry that is provided and configured to be gripped by an external handling device (not shown). The sterile goods container 1 can be handled/moved/manipulated in its position via the external handling device via the handling portion 11. The tag receptacle or the tag 9 in the tag receptacle are configured in a geometrically defined arrangement relative to the handling portion 11. In other words, the handling portion 11 is configured in a defined distance and geometric arrangement to the tag 9 in a virtual coordinate system, wherein these can be mathematically represented in relation to each other by rotational and translational operations, so that optical systems that interact with the handling device in particular in terms of software can detect and control the exact position of the handling portion 11.

FIG. 2 shows an enlarged section A of FIG. 1. In particular, FIG. 2 shows the tag receptacle in the front plate 7 with the tag 9 force-fittingly and/or form-fittingly fixed therein. The tag 9 contains various elements on a surface facing away from the front plate 7, such as an optically readable marker 13, an optically machine-readable QR code 15, a through hole 17 for representing elements located behind the tag 9 on the front plate 7, alphanumeric information 19 and further information about the manufacturer 21. This surface can be referred to as the front side of the tag 9. The arrangement of the various elements shown in this configuration example is to be understood as an example. Of course, other arrangements of the elements on the tag 9 are possible. The optically readable marker 13 enables automated handling of the sterile goods container 1 and also serves to inform the user about this functionality. In other words, a user can recognize from the optically readable marker 13 that the sterile goods container 1 in question is suitable for automated handling via an external handling device. The optically readable marker 13 may vary in color and is ideally high-contrast and unambiguous for a recognition device preferably configured with an AI. In other words, the optically readable marker 13 is asymmetrical and not designed as a barcode, data matrix code or the like. The optically readable marker 13 is arranged on the tag 9 in such a way that it is not covered by any components of the sterile goods container 1 and is clearly recognizable.

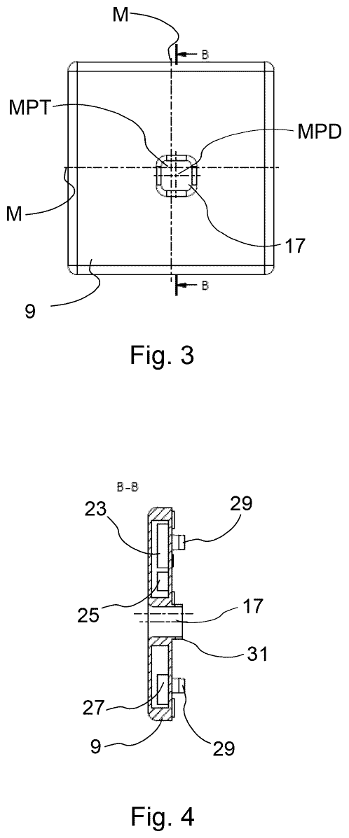

FIG. 3 shows the tag 9 schematically, wherein a representation of the optically readable marker 13, of the optically machine-readable QR code 15, of the alphanumeric information 19 and of further information on the manufacturer 21 has been omitted for better illustration. Furthermore, center marking lines M are shown in FIG. 3 for better illustration. The center marking lines M intersect in a center MPT of the tag 9. A center MPD of the through hole 17 is also shown. The center MPT of tag 9 and the center MPD of the through hole 17 are not aligned. In other words, the through hole 17 is configured off-center in relation to the tag 9. The tag 9 has a rectangular, in particular a square, base area.

FIG. 4 shows a section along line B-B in FIG. 3. An electronic control unit 23 is configured in the tag 9. The electronic control unit 23 includes a radio-based tracking unit which communicates with an external device actively and/or passively, preferably via NFC, Bluetooth Low Energy, DECT or WLAN. Furthermore, the electronic control unit 23 includes a data transmission unit that is provided and configured to transmit data wirelessly. The electronic control unit 23 transmits the data to a control device of the external handling device, for example. A disconnecting switch 25 is additionally configured in the tag 9, which is provided and configured to detect a connection and/or disconnection of the tag 9 to/from the tag receptacle of the front plate 7. The disconnecting switch 25 is connected to the electronic control unit 23. The electronic control unit 23 can, for example, wirelessly transmit the disconnection of the tag 9 from the tag receptacle of the front plate 7 detected by the disconnecting switch 25 as a disconnect signal. The disconnecting switch 25 may be configured as a magnetic switch, for example. An energy storage 27 is also configured in the tag 9. The energy storage 27 is provided and configured to supply the electronic control unit 23 and the disconnecting switch 25 with energy.

The tag 9 is further configured with latching hooks 29 which fix the tag 9 form-fittingly in the tag receptacle of the front plate 7. The latching hooks 29 extend substantially vertically away from a rear side of the tag 9. The rear side of the tag 9 is a side which, in a state in which the tag 9 is mounted in the tag receptacle of the front plate 7 of the sterile goods container 1, lies against the tag receptacle of the front plate 7. The rear side is opposite the front side. Furthermore, a latching-hook extension 31 extends substantially vertically away from the tag 9 at the rear side of the tag 9. The latching-hook extension 31 substantially continues an inner surface of the through hole 17. Like the through hole 17, the latching-hook extension 31 is arranged off-center from the tag 9 in a plan view.

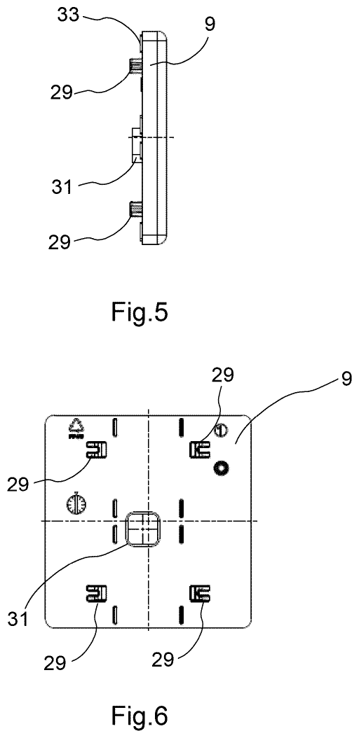

FIG. 5 shows a side view of the tag 9. The tag 9 has a substantially disk-shaped or tile-like shape. In other words, the height of the tag 9 is small compared to its surface area. The height is to be understood as a distance between the front side and the rear side of the tag 9. The latching hooks 29 and the latching hook extension 31 protrude from the tag 9 in the height direction of the tag 9. On the rear side of the tag 9, latching aids 33 are configured around the circumference of the tag 9.

FIG. 6 shows the rear side of the tag 9 with the latching-hook extension 31 and the latching hooks 29. The latching hooks 29 are each configured in the corner portions of the tag 9 in order to ensure that the tag 9 is fixed in the tag receptacle as flat as possible. Due to the off-center positioning of the latching hook extension 31 in conjunction with the positions of the latching hooks 29 in the corner portions of the tag 9, the tag 9 can only be held in exactly one position and orientation in the tag receptacle of the front plate 7. This ensures that the optically readable marker 13 is positioned at the position provided for it in relation to the geometry of the sterile goods container 1. In other words, the latching hooks 29 and the latching-hook extension 31 are arranged asymmetrically.

In the following, an exemplary mode of operation, in particular of the system of the disclosure, is explained in more detail with reference to the Figures. In particular, an automated handling process of the sterile goods container 1 is described.

A first external device preferably detects the position of the radio-based tracking unit configured in the electronic control unit 23 of the tag 9 using Bluetooth low energy beacon technology. A second external device detects the position and orientation of the optically readable marker 13 on the surface of the tag 9. In an optional step, a comparison device may compare the position of the tag 9 determined by the first external device with the position of the tag 9 determined by the second external device. The comparison device may be AI-based. From the determined position of the tag 9, a position of the handling portion 11 is mathematically determined in a processing unit via predetermined rotational and translational operations (e.g. stored by a user). The handling device grips the sterile goods container 1 at the handling portion 11 and then handles it.

If the disconnecting switch 25 detects that the tag 9 is disconnected from the tag receptacle, the electronic control unit 23 may send a stop command to the handling device or an associated external control device so that a collision can be prevented.

In an alternative embodiment, the tag 9 may be configured with photovoltaic elements on the front side and/or on an upper side facing the deck, which supply the electronic control unit 23 and the disconnecting switch 25 with electrical energy either directly or by using the energy storage 27 as a buffer store. A configuration of piezo elements for energy generation on the tag 9 is also conceivable.

In a further alternative embodiment, the tag 9 may include a speaker and/or a piezo buzzer and/or a display and/or LEDs that can output a status of the tag 9. For example, the tag 9 may flash and/or emit an alarm sound if the disconnecting switch 25 detects a disconnection of the tag 9 from the tag receptacle or from the sterile goods container 1. Alternatively or additionally, this may make it easier to locate a specific sterile goods container 1 in a warehouse or the like. In this way, a user can wirelessly control the respective tag 9 and the electronic control unit 23 can trigger a locating alarm via the loudspeaker and/or the display and/or the LEDs.

In another alternative embodiment, the tag 9 may be provided and configured to be inserted into an insertion device on the sterile goods container 1.

In a further embodiment, the tag 9 may have at least one switch that can be used to switch special electronic functions of the tag 9 on and off. For example, the switch may be used to deactivate the locating alarm.

In a further embodiment, the tag 9 may include a mechanical sliding element with which a certain status can be made recognizable manually. This can be done, for example, by revealing a certain color and/or text.

### LIST OF REFERENCE SIGNS

1 sterile goods container
3 container body
5 lid
7 front plate
9 tag
11 handling portion
13 optically readable marker/marker element
15 optically machine-readable QR code
17 through hole
19 alphanumeric information
21 information on the manufacturer
23 electronic control unit
25 disconnecting switch
27 energy storage
29 latching hook
31 latching-hook extension
33 latching aid
M center marking line
MPT center of the tag
MPD center of the through hole

1-13. (cancelled)

14. A tag for determining a location and/or tracking the location and/or determining an orientation of a medical product, the tag comprising:
at least one radio-based tracking unit configured to communicate wirelessly with at least one first external device actively and/or passively; and
at least one optical marker element configured to provide information that is optically detectable by at least one second external device with respect to a position and orientation of the at least one optical marker element in a virtual coordinate system.

15. The tag according to claim 14, wherein the at least one radio-based tracking unit is configured to communicate wirelessly with the at least one first external device actively and/or passively via NFC, Low Energy Bluetooth, DECT or WLAN.

16. The tag according to claim 14, wherein the tag is configured with at least one machine-readable data element and/or at least one alphanumeric data element.

17. The tag according to claim 14, wherein the tag includes at least one optical signal output device.

18. The tag according to claim 17, wherein the at least one optical signal output device comprises:
LEDs; and/or
ePaper displays or other types of displays; and/or
an acoustic signal output device.

19. The tag according to claim 14, wherein the tag is configured with a through hole in a direction normal to a planar extension of the tag.

20. The tag according to claim 14, wherein the tag is configured with at least one energy generating element.

21. The tag according to claim 20, wherein the at least one energy generating element is a photovoltaic element or a piezo element.

22. The tag according to claim 14, wherein the tag is configured with at least one manual status sliding element.

23. A medical product comprising:
a tag according to claim 14; and
a tag receptacle that receives the tag in a predetermined position and orientation with respect to the medical product or a part thereof.

24. The medical product according to claim 23, wherein a disconnecting switch is configured between the tag and the tag receptacle of the medical product, the disconnecting switch configured to detect a disconnection of the tag from the medical product.

25. The medical product according to claim 24, wherein the medical product includes a disconnect signal transmitter unit that transmits a wireless disconnect signal when the disconnecting switch detects the disconnection of the tag from the medical product.

26. A system for determining a location and/or tracking the location and/or determining the orientation of a medical product, the system comprising:
the tag according to claim 14;
at least one first external device that wirelessly communicates actively and/or passively with the tag and determines a position of the tag based on a range and/or a signal strength of at least one wireless signal and/or based on lateration of the at least one wireless signal; and
at least one second external device that optically detects a position and an orientation of the tag via the at least one optical marker element.

27. The system according to claim 26, wherein the position of the tag is determined based on a trilateration or a multilateration.

28. The system according to claim 26, wherein the system includes at least one handling device configured to grip and handle the medical product at a handling portion.

29. The system according to claim 28, further comprising a processing unit configured to determine a position of the handling portion from the position of the tag based on geometric dependencies stored in the processing unit.

30. The system according to claim 26, further comprising a comparison device that compares a first position signal determined by the at least one first external device with a second position signal determined by the at least one second external device.
